(19) United States
(12) Patent Application Publication
(10) Pub. No.: US 2009/0209882 A1
(43) Pub. Date: Aug. 20, 2009

(54) INTEGRATED CAP AND SAMPLE APPLICATOR

(75) Inventor: Alex M. Saunders, San Carlos, CA (US)

Correspondence Address: HOVEY WILLIAMS LLP, 10801 Mastin Blvd., Suite 1000, Overland Park, KS, 66210, US

(73) Assignee: Trinity Research Limited, Bray (IE)

(21) Appl. No.: 12/032,209

(22) Filed: Feb. 15, 2008

(51) Int. Cl. A61B 5/151

(52) U.S. Cl. 600/573

(57) ABSTRACT

A sampling cap (10) for a cuvette (72) or other liquid sampling device is provided, which includes a cap body (12) having a fixedly mounted, axially extending capillary collection tube (40). The body (12) includes first and section end sections (14, 16) with in an intermediate shoulder (18). Each of the sections (14, 16) is equipped with friction fitting ribs (24, 30). In use, the second end section (16) of cap body (12) is positioned within the open end of a holder (68) or cuvette (72), and the assembly is manipulated to cause a fluid sample (e.g., blood) to be drawn into capillary tube (40). The cap body (12) is then detached and inverted, and the first end section (14) is pressed into the open end (70) of cuvette (72) with capillary tube (40) thereby located within the confines of the cuvette (72). The liquid sample within the capillary tube (40) is then discharged into the cuvette (72) by shaking or the like.

### BACKGROUND OF THE INVENTION

1. Field of the Invention

The present invention is broadly concerned with a sampling cap for use with a cuvette or similar sample collection device, in order to facilitate collection and analysis of fluid samples. More particularly, the invention is concerned with such a sampling cap and method of use thereof, wherein the cap includes an end-mounted, fixed capillary tube operable to collect a liquid sample, with an adjacent end section permitting the sample-filled cap to be inverted and fitted within the open end of the collection device.

2. Description of the Prior Art

Many biological fluids are analyzed using spectrophotometric techniques. For example, samples of blood may be drawn, mixed with appropriate reagents in a cuvette, and then analyzed. The conventional method of obtaining small blood samples is to prick the finger or earlobe of a patient using a lancet, followed by collecting the blood sample using a micropipette or capillary tube. Once collected, the filled capillary tube maybe dropped into an open cuvette, followed by capping of the cuvette. Thereafter, the sample is discharged from the capillary tube by shaking or tumbling. In some instances, and depending upon the nature of the liquid sample, the capillary tube may need pretreatment with a surface active agent to facilitate sample wicking into the capillary tube, or to provide an anticoagulant coating on the tube to prevent clotting of blood therein. This type of sample manipulation thus requires multiple steps and may be somewhat difficult for untrained personnel.

A number of different cuvette or other sampling systems have been disclosed in the prior art. For example, U.S. Pat. No. 3,794,469 discloses a blood testing system including a cuvette cap which is designed to receive a separate, sample-filled micropipette. Once installed on a cuvette, the contents of the micropipette are discharged into the cuvette. Similarly, U.S. Pat. No. 5,651,940 describes a cuvette tube cap equipped with a through-aperture for receiving a sample tube. Sealing means are provided in the cap to normally seal the aperture. U.S. Pat. No. 5,128,104 describes specialized cuvettes having hingedly mounted covers. Finally, U.S. Pat. No. 5,833,630 discloses a sample collection device including a cuvette with a stopper, the latter having a permanently attached capillary extending therethrough. A pressure cap is applied over the upper end of the stopper and capillary tube and is pressed downwardly to expel a collected sample into the cuvette. In general, these devices simply add to the complexity of fluid sample collection, rather than facilitating the operation.

There is accordingly a need in the art for an improved sampling cap for a cuvette or similar container which allows quick, easy sample collection without the complications and specialized structures characteristic of the prior art.

### SUMMARY OF THE INVENTION

The present invention overcomes the problems outlined above, and provides a sampling cap for a liquid collection device such as a cuvette. The cap comprises a cap body including a first end section configured to frictionally fit within and seal the open end of a cuvette, and a capillary tube mounted on the first end section and oriented to be within the cuvette, when the cap body first end section is fitted within the open end of the cuvette. The capillary tube is operable to collect and hold a liquid sample, and to permit the discharge of the collected sample into the cuvette by shaking or similar agitation. The cap is preferably formed a part of a cap assembly including a flexible cover disposed over the capillary tube and a base, these components serving to seal the cap and capillary tube.

In preferred forms, the first end section of the cap body presents an end surface, with the capillary tube fixedly mounted in spaced relationship to the end surface. For this purpose, a pair of oblique mounting struts are provided to support tie capillary tube above the end surface. Although not essential, it has been found that the capillary tube should be substantially C-shaped in plan, with an elongated slot formed along the length of the tube. This configuration facilitates wicking of the liquid sample into the capillary tube, and also discharge into a cuvette.

The preferred cap body also includes a second end section with an intermediate shoulder between the end sections. Both end sections have a series of circumferential ribs assuring a tight friction fit of the end sections into appropriately sized cuvettes or holders.

In practice, the second end section of the cap body is pressed into a tubular holder or the open end of a cuvette tube, with the capillary tube extending upwardly. This assembly is then manipulated and held at an appropriate angle so that a liquid sample such as blood is drawn into the capillary tube. At this point, the cap is removed and inverted so that the capillary tube is located within the confines of a cuvette. The cap is pressed into the cuvette open end until a firm friction fit is established, whereupon the liquid sample is discharged into the cuvette by shaking or the like.

### BRIEF DESCRIPTION OF THE DRAWINGS

FIG. 1 is a perspective view of the preferred cuvette sampling cap of the invention;

FIG. 2 is a is a side elevational view of the cap;

FIG. 3 is a is a plan view of the cap;

FIG. 4 is an exploded perspective view of the complete cuvette sampling cap assembly;

FIG. 5 is a vertical sectional view of the assembly illustrated in FIG. 4;

FIG. 6 is a greatly enlarged, fragmentary sectional view illustrating the construction of the base of the cap;

FIG. 7 is an elevational view of the sampling cap assembly mounted on a tubular holder;

FIG. 8 is a perspective view illustrating use of the sampling cap during collection of a blood sample; and

FIG. 9 is a view in partial vertical section illustrating the inverted installation of the cap on a cuvette.

### DETAILED DESCRIPTION OF THE PREFERRED EMBODIMENT

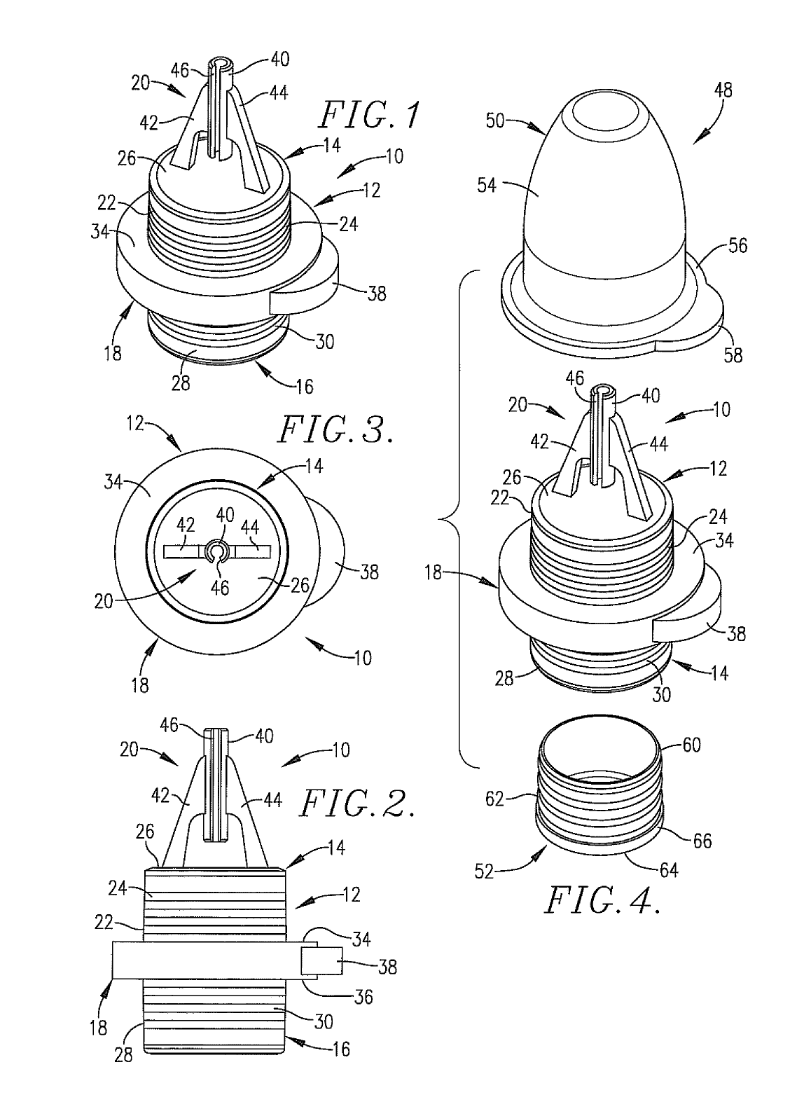

Turning now to the drawings, a cuvette sampling cap 10 is illustrated in FIGS. 1-3. The cap 10 includes an elongated cap body 12 presenting a first tubular end section 14, a second tubular end section 16, and a central, radially outwardly extending shoulder 18. Additionally, the cap 10 includes an axially extending capillary assembly 20 permanently and fixedly mounted on section 14.

In more detail, the first end section 14 includes a tubular sidewall 22 with a series of slightly radially expanded circumferential ribs 24. The section 14 also has a top wall 26. The second end section 16 also includes a tubular sidewall 28 having the same type of circumferential ribs 30. As best seen in FIGS. 5 and 6, the second end section 16 presents an open end 32 having an annular abutment surface 33.

The shoulder 18 is located midway along the length of the cap body 12, between the first and second ends section 14 and 16. The shoulder 18 is radially expanded and presents opposed annular stop surfaces 34, 36. Also, an arcuate projection 38 is provided on shoulder 18 to facilitate manual manipulation of the cap 10 as will be described.

The capillary assembly 20 includes an elongated capillary tube 40 located in spaced relationship above top wall 26, together with a pair of obliquely oriented mounting struts 42, 44. As illustrated the struts 44 are secured to top wall 26 and serve to support capillary tube 40. It will be seen that the tube 40 does not extend through top wall 26 and into the confines of first end section 14. It will also be observed that the tube 40 is of somewhat C-shaped configuration in plan, having an elongated, axially extending slot 46 extending the full length of the tube 40.

The cap 10 is normally provided as a part of a cap assembly 48, best seen in FIGS. 4 and 5. The overall assembly 48 includes the cap 10, as well as a flexible top cover 50 and a lower tubular base member 52. The cover 50 has an upper bonnet section 54, as well as a lower most, radially outwardly extending lip 56 designed to abut stop surface 34. The lip 56 also has a projection 58 designed to overlie shoulder projection 38. Referring to FIG. 5, it will be seen that the bonnet 54 engages the outer surface of sidewall 22 to provide a seal, and is sized to accommodate the capillary assembly 20.

The base member 52 is of tubular design, having an upwardly extending sidewall 60 equipped with slightly outwardly projecting, circumferential ribs 62, and a bottom wall 64 having a radially outwardly extending shoulder section 66. As illustrated, the base member 52 is sized to fit within the lower open end 32 of second end section 16. Specifically, the member 52 is pushed into the end 32 until the shoulder section 66 abuts surface 33, and with the ribs 62 engaging the inner surface of sidewall 28, thereby providing a seal.

It will thus be appreciated that the overall cap assembly 48 provides a sealed, substantially air-tight enclosure about capillary assembly 20, in order to maintain the sterility thereof and to prevent entrance of contaminants.

The cap 10 and base member 52 are preferably formed by injection molding or other suitable technique, using conventional synthetic resin materials. The cover 50 is normally fabricated from a flexible synthetic resin.

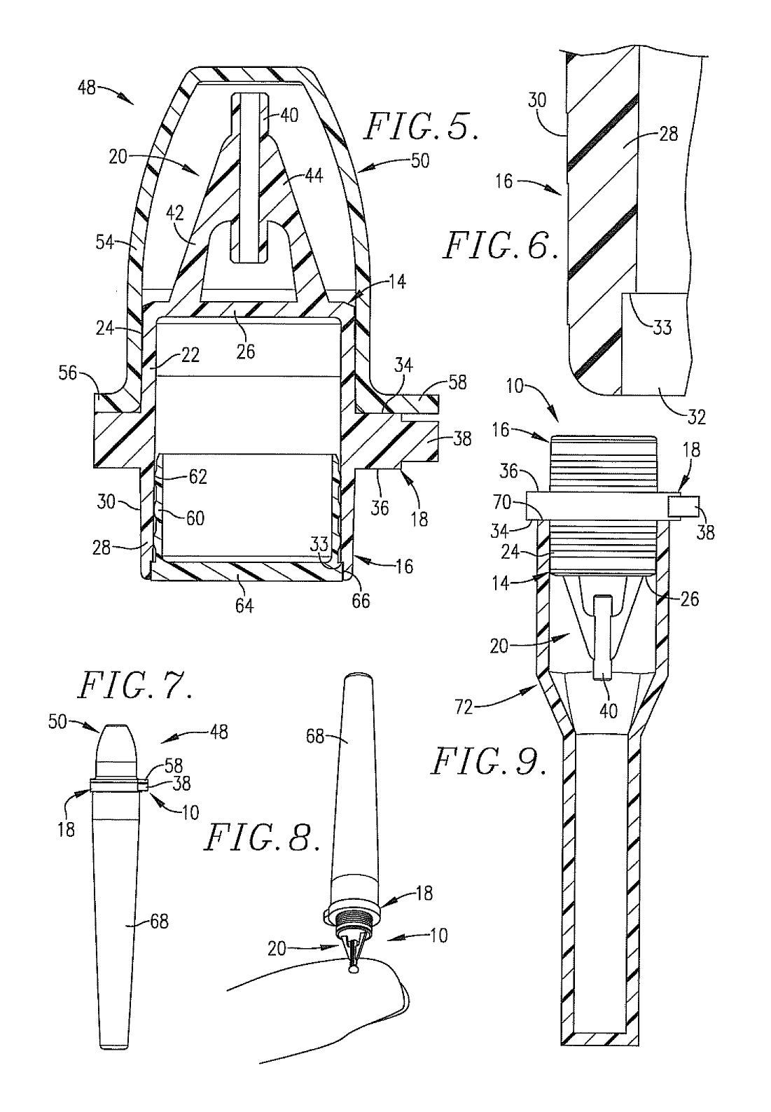

FIGS. 7-9 illustrate an exemplary use of the cap assembly 48 and cap 10. Referring to FIG. 7, it will be seen that the assembly 48 is mounted on a tubular holder 68. Specifically the second end section 16 of the cap 10 is inserted into the upper end of holder 68 until the shoulder 18 abuts the upper end of the holder, with the peripheral ribs 30 serving to establish a secure, friction fit within the holder. In the next step, the cover 50 is removed by grasping the lip projection 58 and gently pulling the cover upwardly and removing it from the first end section 14. At this point the capillary assembly 20 is ready for use. As depicted in FIG. 8, a liquid blood sample, drawn by a lancet (not shown) is drawn upwardly into tube 40 by capillary action until the tube is filled. The provision of slot 46 allows the sample to be taken more readily than with a conventional closed sidewall capillary tube. The holder 68 is next inverted above the open end 70 of a cuvette 72 (FIG. 9), and the first end section 14 of the cap body 12 is forced into the open end 70 until the surface 34 of shoulder 18 comes into abutment with the upper most end of the cuvette. Again, this establishes a frictional sealing fit between the cap 10 and cuvette 72, owing to the engagement of ribs 24 with the inner surface of cuvette 72. In the final step, the cuvette 72 is shaken or otherwise agitated, causing the liquid sample within tube 40 to be deposited into the body of the cuvette 72, which also typically contains other liquid reagents. The cuvette 72 can then be analyzed using a spectrophotometer or other conventional analytical device.

It will also be appreciated that the cap 10 can be used with a single cuvette 72, without need for the holder 68. In such a use, the second end section 16 of the cap is first placed within the open end of the cuvette 72 so that shoulder surface 36 abuts the end of the cuvette, and with the assembly 20 projecting upwardly. The cuvette 72 is then used in the same fashion as holder 68 to draw a liquid sample into tube 40. Thereupon, the cap 10 is detached from the cuvette 72 by grasping projection 38 and pulling the cap out of the open end 70. The cap 10 is then inverted and the first end section 14 is inserted into the open end 70 of the cuvette 72 until shoulder surface 34 abuts the end of the cuvette. At this point the liquid sample within tube 40 is dislodged as previously described, and the cuvette can be analyzed.

The present invention is particularly suited for use with cuvette tubes used in the single-container optical assays described in Published U.S. Patent Application 2007/0264718, which is incorporated by reference herein.

1. A sampling cap for a liquid collection device, comprising:
a cap body including a first end section configured to frictionally fit within and seal the open end of a liquid collection device; and
a capillary tube mounted on said first end section and oriented to be within said device when the cap body first end section is fitted within the open end of the device,
said capillary tube having a pair of opposed, open ends, and an elongated opening extending the full length of the capillary tube and communicating with said open ends, said elongated opening having a substantially constant width throughout the length thereof,
said capillary tube operable to collect and hold a liquid sample, and to permit the discharge of the collected sample into the device.

2. The sampling cap of claim 1, said cap body first end section presenting an end surface, said capillary tube located in spaced relationship to said end surface.

3. (canceled)

4. The sampling cap of claim 1, said cap body including an outwardly extending shoulder spaced from said end surface and oriented to engage the upper end of said device.

5. The sampling cap of claim 4, including a projection extending outwardly from said shoulder to facilitate manipulation of the cap body.

6. The sampling cap of claim 1, including a second end section also configured to frictionally fit within and seal the open end of the device.

7. The sampling cap of claim 6, said first and second end sections having outwardly projecting ribs configured to engage the inner surface of the open end of said device.

8. A method of collecting and handling a fluid sample, comprising the steps of:
providing a sampling cap having a capillary tube mounted thereon, said capillary tube having a pair of opposed, open ends, and an elongated opening extending the full length of the capillary tube and communicating with said open ends, said elongated opening having a substantially constant width throughout the length thereof;
manipulating said sampling cap so as to cause a liquid sample to be drawn into said capillary tube;
after said manipulating step, positioning said sampling cap within the open end of a liquid collection device, and with said capillary tube extending into the device; and
discharging said sample into said device.

9. The method of claim 8, said sample discharging step comprising agitating the device until said sample is discharged.

10. The method of claim 8, said manipulating step comprising the steps of mounting said sampling cap onto a holder, and locating said capillary tube at an appropriate angle for causing said liquid sample to be drawn into the capillary tube.

11. The method of claim 8, said positioning step of inverting said cap and frictionally fitting the cap within said open end of said device in order to seal the device.
